SEQUENCE LISTING (3 sequences)

SEQ ID NO: 1 - protein, 46 aa, Artificial Sequence
  Modified Human Peptide
CGKRDAEFRHDSGYEVHHQKLVFFAEDVGSNKGAIIGLMVGGVVIA

SEQ ID NO: 2 - protein, 42 aa, Homo sapiens [sequence omitted]

SEQ ID NO: 3 - protein, 41 aa, Artificial Sequence
  Modified human peptide
CDAEFRHDSGYEVHHQKLVFFAEDVGSNKGAIIGLMVGGVV

(19) United States
(12) Patent Application Publication
(10) Pub. No.: US 2013/0109033 A1
(43) Pub. Date: May 2, 2013

(54) Method, Particularly Enzyme-Linked Immunosorbent Assay (ELISA), for In Vitro Detection of Amyloid Beta Autoantibodies, Microtiter Plate, and Test Kit

(71) Applicants: Michael Bacher, Colbe (DE); Richard Dodel, Niederweimar/Lahn (DE); Karthikeyan Balakrishnan, Ludwigshafen (DE)

(72) Inventors: Michael Bacher, Colbe (DE); Richard Dodel, Niederweimar/Lahn (DE); Karthikeyan Balakrishnan, Ludwigshafen (DE)

(73) Assignee: Philipps-Universitat Marvurg, Marburg (DE)

(21) Appl. No.: 13/384,726

(22) PCT Filed: Jul. 14, 2010
(86) PCT No.: PCT/DE10/00817

§ 371 Date: May 7, 2012

(30) Foreign Application Priority Data
Jul. 20, 2009 (DE) 10 2009 034 119.6

(51) Int. Cl. G01N 33/53

(52) U.S. Cl. CPC G01N 33/5306
USPC 435/7.92; 435/287.2

(57) ABSTRACT

A method, particularly an enzyme-linked immunosorbent assay (ELISA), for the in-vitro detection of Aβ autoantibodies in human serum and/or plasma contains the following steps: preparing an antigen-coated solid phase; incubating the solid phase with a blocking solution; incubating the solid phase with a sample to be examined; immunological detection of the Aβ autoantibodies on the solid phase; and reading the detected results of the solid phase using a reading tool. According to the invention, the preparation of the antigen-coated solid phase advantageously includes incubating the solid phase with a coating solution in which the antigen is dissolved, said antigen having a peptide sequence selected from the group SEQ ID No. 1, SEQ ID No. 2 or SEQ ID No. 3.

The invention concerns a method, in particular an enzyme-linked immunosorbent assay (ELISA), for the in-vitro verification of amyloid-beta auto-antibodies, according to the general terms of claim 1, a microtiter plate according to claim 17, and a test kit according to claim 23.

In-vitro verification methods for antibodies, particularly ELISAs, are generally known. They belong to the group of immunoassay methods. Their basic principle is the recognition of an analyte by its binding partner in the form of binding an antibody to an antigen. The following is to be understood, in the sense of the present application:

| | |
|---|---|
| Analyte: | An antigen to be verified or an antibody to be verified in a |
| | sample to be studied |
| Antigen: | A protein/polypeptide that causes the production of anti- |
| | bodies when it is injected into an animal organism (antigen |
| | stimulus) |
| Antibody: | A protein that is produced as a reaction to the antigen |
| | stimulus and specifically recognizes and binds the antigen |
| | producing the stimulus |
| Anti-species | An antibody that is produced when proteins (including |
| antibody: | antibodies) of one species are injected into other species |
| | and they recognize and bind all antigens that originate |
| | from the first species |
| Binding partner: | An antigen or antibody that binds specifically to the |
| | analyte. |
| Detection | Is bound to the analyte, bur not to the binding partner of |
| antibody: | the analyte and is either coupled (=conjugated) with a |
| | detection means or with an immediately verifiable |
| | substance, such as, for example, a fluorescent dye. With |
| | the detection means, there may be involved, for example, |
| | an enzyme which is in a position to break down a special |
| | substrate, such that a color reaction is produced. In the |
| | sense of the present invention, the detection antibody can |
| | also be described as a second antibody. |

The procedure in such a verification method is basically represented as follows:

The binding partner of the analyte is immobilized in a fixed phase. Then the fixed phase is incubated with a sample in which the analyte to be verified might be found. Provided that the analyte is present in the sample, it binds to the binding partner immobilized in the fixed phase and is likewise immobilized in this way. The binding of the analyte to its binding partner is then verified with the aid of a detection antibody. The fixed phase, to which the binding partner of the analyte is now fixed at the same time, is incubated with a solution that contains the detection antibody. This binds to the immobilized analyte, and it can then, for example, be verified by carrying out an enzyme-substrate reaction with the aid of the enzyme conjugated to the detection antibody.

Such verification methods are preferred to be used in medical diagnostics, in order to be able to examine samples of tissue or body fluids from an animal and/or human being for the presence or absence of antigens. Conclusions can then be drawn from the result of the verification statements, for example, concerning possible diseases of the examinee.

A prominent example of diseases that can be studied in this way is Alzheimer's disease. This disease is characterized by a series of neuropathological features, of which a particularly typical one is the formation of so-called neuritic plaques in the brain of the patient. These plaques consist of extracellularly deposited amyloid-beta peptides (Aβ), which are formed upon the breakdown of amyloid precursor proteins (APP) [Kang et al., “The precursor of Alzheimer's disease, amyloid A4 protein, resembles a cell-surface receptor”, Nature (1987); Tanzi et al., “Amyloid-beta protein gene: cDNA, mRNA distribution, and genetic linkage near the Alzheimer locus”, Science (1987)].

It is problematic, with conventional ELISAs for Aβ verification that not all Aβ forms are actually pathological, and that antibodies that specifically recognize the pathological forms are available in only a very limited range and are accordingly very expensive. Thus, two at least two specific antibodies are needed for verification: a first one as the binding partner immobilized to the fixed phase and a second one as a detection antibody. These should accordingly also originate from different species, because the detection otherwise holds the danger of a very high background and accordingly the danger of giving a high number of false positive results.

The formation of a non-specific background can admittedly be reduced so that the detection antibody not binding directly to the bound Aβ-peptide but to a second antibody that recognizes and binds the bound analyte but is not a conjugated enzyme. However, this also has disadvantages. Thus, for this, comparatively many antibody-antigen binding steps are necessary: capturing the Aβ-peptide to be verified by a first antibody bound on the plate, binding the second antibody to the captured antigen, binding the second antibody by means of the detection antibody, which does not actually reduce the likelihood of errors and therewith the chance of false positive or false negative test results.

Du et al. however observed in 2001 that natural antibodies to Aβ-peptide, so-called amyloid-beta auto-antibodies (Aβ-auto-antibodies), are present in body fluids of both healthy individuals and Alzheimer patients, [cf. Du et al., “Reduced levels of amyloid β-peptide antibody in Alzheimer disease”, Neurology (2001)]. What is more, the titer of these so-called auto-antibodies changes if Alzheimer's disease is present.

This observation can in principle be exploited to perform a modified ELISA. The auto-antibody is then verified instead of an Aβ-peptide. For this, either samples of cerebrospinal fluid (CSF) or blood serum or plasma samples are taken and examined. Both of these however are associated with clear drawbacks. So on the one hand, taking CSF samples is not unproblematic and is unpleasant for the patient. On the other hand, the analysis of serum samples, which can be easily obtained, in contrast, is possible only at greatly increased expense. Thus, a particular difficulty lies in the enormous general antibody density in the serum matrix, which hinders specifically picking out the Aβ auto-antibodies by means of the corresponding binding partner immobilized in the fixed phase of the test and frequently causes non-specific bindings or unwanted cross-reactions.

The task of the invention is therefore to make available an improved ELISA for the verification of Aβ auto-antibodies, which allows rapid evaluation by simple and cost-effective means. The ELISA should, particularly with human serum and/or plasma samples and CSF samples, be fast and simple to perform. Furthermore, a task is to provide an appropriate microtiter plate as well as a test kit to perform an ELISA according to the invention.

The main features of the invention are stated in the characterization portion of claims 1, 17, and 23. Embodiments are the subject of claims 2 through 16, 18 through 22, 24, and 25.

With a method, in particular an enzyme-linked immunosorbent assay (ELISA), for in-vitro verification of amyloid-beta auto-antibodies in human blood serum and/or plasma, comprising the steps
a) preparation of an antigen-coated fixed phase,
b) incubation of the fixed phase with a blocking solution,
c) incubation of the fixed phase with a sample to be examined,
d) immunological verification of the amyloid-beta auto-antibody in the fixed phase, and
e) readout of the verification results from the fixed phase using a reader,
   the invention provides that the preparation of the antigen-coated fixed phase includes the incubation of the fixed phase with a coating solution in which an antigen is dissolved which exhibits a peptide sequence selected from the group SEQ ID No. 1, SEQ ID No. 2, or SEQ ID No. 3.

At the same time, it has, in particular, been proven to be an advantage if the fixed phase is coated with an antigen that has a peptide sequence corresponding to one of the sequences SEQ ID No. 1, SEQ ID No. 2, or SEQ ID No. 3. Surprisingly, such antigens, specifically peptides 40 to 46 amino acids long, show very rapid aggregation kinetics with natural human Aβ auto-antibodies. They therefore rapidly and reliably bind auto-antibodies to be verified in the ELISA to the antigens immobilized in the fixed phase. Moreover, it is especially favorable if the antigen exhibits a peptide sequence corresponding to SEQ ID No. 1 or SEQ ID No. 2.

An especially critical point in setting up an ELISA is, as has already been explained above, the coating of the fixed phase with the binding partner of the analyte. A further particular advantage of the invention lies in the fact that the coating solution is a carbonate buffer with a basic pH value. Thus, it has been shown specifically that the antigens according to the invention clearly bind to the fixed phase better in a basic medium, particularly with a pH value of 9.6, than in the conventionally used neutral medium with a pH of about 7.

The incubation of the fixed phase with the coating solution, then, can take place overnight at 4° C., which is especially favorable when using an antigen with a sequence corresponding to SEQ ID No. 1, and at 37° C. and 5% CO₂ for 2 hours, which is the preferred case when using an antigen with a sequence corresponding to SEQ ID No. 2.

A further critical point in performing a verification method according to the invention is blocking. This step serves here to cover up the area of the fixed-phase surface to which no antigen is bound, so that the analyte to be verified can itself bind exclusively to the antigen and not to the fixed phase in the subsequent incubation with the sample. It is preferred, moreover, that the blocking solution be a Tris-buffered protein solution with a pH of 7.4, containing at least one antimicrobial agent. Thus, for example, the commercially available blocking solution Superblock TBS® or Superblock® from the Pierce Company Germany, can be used. The incubation with the blocking solution is preferably performed overnight at 4° C. In this way, the free surface of the fixed phase can be covered particularly well with the protein contained in the blocking solution at the sites to which no antigen is bound. Thus, antigens or antibodies present in the sample are effectively prevented from binding non-specifically to the fixed phase and thus giving unwanted false positive signals, which are also indicated as non-specific, disturbing background.

When performing an immunological assay for diagnostic purposes, it has proven that not always is just an individual patient sample to be analyzed, but a standardized comparison sample simultaneously (indicated hereinafter as a standard), which is actually to be analyzed for the patient sample to be examined and at least one control under identical conditions. It is therefore advantageous if the fixed phase is a microtiter plate. This usually has a plurality of sections, so-called wells, for analysis of patient samples and/or a standard and a control. All the wells of a microtiter plate can be identically prepared. Usually, all the wells are simultaneously coated for this with antigen and then blocked. Then the respectively identified wells are filled with a defined amount of standard, patient sample, and control and handled further. The same volume of fluid is filled into each well. The respective concentration of standard, patient sample, and control is, however, varied from well to well by means of suitable dilution, in order to allow as precise as possible a determination of concentration of the analyte to be verified.

A further advantage of the invention lies in the fact that the wells are filled with a relatively high total volume, namely up to 300 μl, which, however, contains only a relatively small share in the sample (that is, either standard, patient sample, or control).

So an embodiment of the verification method according to the invention provides that the wells are first filled with 250 μl of an assay buffer. In this buffer, only 10 μl of the sample to be examined are then added. At the same time, the assay buffer serves in the specified dilution of the sample. In particular, in the analysis of patient samples obtained from serum, it is advantageous if only a small volume of serum has to be used for the verification. This minimizes unwanted cross-reactions and in this way raises the specificity of the test.

What is more, it is also conceivable that serum samples which exhibit a especially high concentration of serum components have already been pre-diluted before introduction into the assay buffer using a so-called sample-dilution buffer. This sample-dilution buffer is preferably made so that the serum matrix is not damaged during the dilution. A further embodiment accordingly provides that the wells are filled with 200 μl of diluted sample.

After filling the well with the sample (that is, with standard, patient sample, and control), the microtiter plate is incubated for 60 minutes in a shaker at 300 to 500 rpm and room temperature. These conditions have been emphasized as especially preferred in order to, on the one hand, ensure that the analytes present in the patient sample actually bind to their binding partner immobilized on the microtiter plate, and on the other hand to stop unwanted cross-reactions or non-specific bindings insofar as possible.

It is recognized that the incubation of the microtiter plate with the solution to be examined preferably includes the following steps: introduction of the diluted sample into each well of the microtiter plate, whereby the introduction of the diluted sample occurs such that a total volume of 200 to 300 μl of the assay buffer containing the sample is then present in each well, and incubation for 60 minutes in a shaker at 300 to 500 rpm and room temperature.

Moreover, the assay buffer preferred for this is a sodium phosphate buffer with a pH of 7.0, including 3 to 9% BSA, 0.01 to 3% TWEEN, and at least one preservative selected from the group of 5-bromo-5-nitro-1,3-dioxane (BND), 2-chloroacetamide (CAA), 2-hydroxypyridine-N-oxide (HPO), N-methyliso-thiazolone (MIT), sodium azide, Thimerosal, and ProClin.

The next step of the method according to the invention is the immunological verification of the Aβ auto-antibody captured by the binding partner in the fixed phase. This occurs by means of the following steps: tapping out the contents of the well, three to five washings of the well with 300 to 500 μl respectively of rinse solution per well, removing remaining fluid drops by wiping the well out with an absorbent paper, introducing 50 μl to 100 μl of enzyme conjugate into each well, incubating for 30 to 60 minutes in a shaker at 300 to 500 rpm and room temperature, shaking the contents out of the well, washing the well three to five times with 300 to 500 μl respectively of rinse solution per well, removing remaining fluid drops by wiping the well out with an absorbent paper, adding 50 μl substrate solution into each well, incubating for 15 to 20 minutes at room temperature, and stopping the enzyme reaction by adding 100 μl stop solution into each well.

The preferred rinse solution for this is a Tris buffer containing 0.01 to 3% TWEEN.

A particular advantage of the ELISA according to the invention lies in the fact that the binding kinetics of the Aβ auto-antibody to the antigen according to the invention is optimal, so that a chromogenic enzyme-substrate reaction can be used for the immunological verification, whose result can be measured by determining the optical density at 450 nm. Accordingly, the readout of the verification result occurs at 450±10 nm. What is more, it is favorable if the readout takes place within 10 minutes of adding the stop solution.

The enzyme conjugate introduced into the well after the first wash procedure contains the detection antibody. Moreover, it preferably involves an antibody targeted against human IgG (α-human IgG). This can, for example, originate from one of the following species: goat, mouse, porpoise, rat, donkey, cow, sheep, or pig. Others are admittedly generally less usual but are also conceivable. The α-human IgG antibody is conjugated with an enzyme or dye, so that it can be made visible, either directly if it is a matter of a fluorescent dye, for example, or by converting a substrate with the aid of the enzyme. With the enzyme, this can involve, for example, an enzyme selected from the group of peroxidase (POD), horseradish peroxides (HRP), alkali phosphatase (AP), and βgalactosidase (β-gal). Further enzymes are imaginable which cause a color reaction with an appropriate substrate. A coupling with biotin or polymers is also conceivable, to which a plurality of enzymes is coupled.

It is recognized that the enzyme conjugate of an antibody targeted against human IgG contains an enzyme conjugate that originates from a species selected from goat, mouse, porpoise, rat, donkey, cow, or sheep, whereby the enzyme conjugated with the antibody is selected from the group of horseradish peroxides (HRP), alkali phosphatase (AP), and β-galactosidase (β-gal).

Each of these enzymes can convert different substrates, so that a color reaction is produced which can be evaluated. The selection of the substrate is accordingly directed toward the selection of the enzyme and vice versa. Suitable substrates can, for example, be selected from the following group: 3,3′-diaminobenzidine (DAB), 3-amino-9-ethylcarbazole (AEC), 4-chloro-1-naphthol (CN), tetramethyl-benzidine (TMB), new fuchsine, naphthol-AS-MX-phosphate, 5-bromo-5-chloro-3-indoxyl phosphate (BCIP), nitro blue tetrazolium chloride (NBT), 5-bromo-4-chloro-3-indoxyl-β-D-galactopyranoside (X-gal), 5-bromo-3-indolyl-6-D-galactopyranoside (blue-gal), 6-chloro-3-indolyl-β-D-galactopyranoside (Y-gal), 5-iodo-3-indolyl-β-D-galactopyranoside (purple-gal), 5-bromo-6-chloro-3-indolyl-β-D-galactopyranoside (magenta-gal), N-methylindolyl-β-D-galactopyranoside (green-gal), 4-methyl-umbelliferyl-β-D-galactopyranoside (MUG). The reaction conditions are then directed respectively toward the enzyme selected. It is preferable if the substrate selected is soluble such that it can be introduced in a volume of 50 to 100 μl into the well of the microtiter plate.

It is known that the substrate solution contains at least one chromophore selected from the group of 3,3′-diaminobenzidine (DAB), 3-amino-9-ethylcarbazole (AEC), 4-chloro-1-naphthol (CN), tetramethylbenzidine (TMB), new fuchsine, naphthol-AS-MX-phosphate, 5-bromo-5-chloro-3-indoxyl phosphate (BCIP), nitro blue tetrazolium chloride (NBT), 5-bromo-4-chloro-3-indoxyl-β-D-galactopyranoside (X-gal), 5-bromo-3-indolyl-β-D-galactopyranoside (blue-gal), 6-chloro-3-indolyl-6-D-galactopyranoside (Y-gal), 5-iodo-3-indolyl-β-D-galactopyranoside (purple-gal), 5-bromo-6-chloro-3-indolyl-β-D-galactopyranoside (magenta-gal), N-methyl-indolyl-β-D-galactopyranoside (green-gal), 4-methylumbelliferyl-β-D-galacto-pyranoside (MUG), in soluble form, which displays a color reaction upon reaction with the enzyme of the enzyme conjugate.

The use of an HRP-coupled α-human IgG antibody in combination with the substrate TMB has shown itself to be especially favorable.

Because the substrate to be broken down by the enzyme is usually added in excess, it is required that the enzyme-substrate reaction be ended controlled after a specified period of time. This can, for example, occur by adding a stop solution. This changes the reaction conditions such that the enzyme-substrate reaction cannot be continued. If the enzyme is, for example HRP and the substrate is TMB, then the reaction is preferably stopped by the addition of an acid, usually sulfuric acid. The stop solution then consists, in the simplest case, of the dilute acid. It is recognized that the stop solution has a pH value less than 7.0 and preferably is a dilute hydrochloric or sulfuric acid.

The invention in addition provides a microtiter plate with at least one well, in which each well is coated with a peptide corresponding to one of the sequences SEQ ID No. 1 to 3. Such a microtiter plate can be used to great advantage in order to perform the method according to the invention.

For an ordinary test design, it is favorable if the microtiter plate exhibits at least one test unit comprising 24 wells. The 24 wells can then be divided into three groups. The first eight wells are loaded with eight different concentrations of the standard. The comparison curves are determined from the measurement values obtained therefrom. The second eight wells are loaded with eight different dilution stages of a serum sample, which would be taken from a healthy control person. The third eight wells are loaded with the corresponding eight dilution stages of a serum sample from a possibly ill person. These three times eight wells together form a test unit.

It is also conceivable that the microtiter plate exhibits several test units, which can be used independently of one another. So, for example, a large microtiter plate with several test units disposed in a linear one behind the other is conceivable, which can be snapped or broken off with the aid of a perforation or groove for the use of the microtiter plate.

To carry out the method according to the invention, thanks to the high sensitivity of the method, both microtiter plates whose wells have a flat bottom and wells with a round, V-shaped, or C-shaped bottom are suitable. It is therefore recognized that with the microtiter plate according to the invention, it is advantageous that the wells have a round, flat, V-shaped, or C-shaped bottom.

It is further known that a microtiter plate according to the invention is made to particular advantage according to a method including the following steps: preparation of an uncoated microtiter plate, incubation of the microtiter plate with a coating solution which consists of a basic carbonate buffer with a pH of 9.6, in which is dissolved 5 μg/ml of an antigen corresponding to one of the sequences according to SEQ ID No. 1, 2, or 3, for two hours at 37°, and incubation of the microtiter plate with a blocking solution containing a Tris-buffered protein solution with a pH of 7.4, containing at least one antimicrobial agent at 4° C. overnight.

It is, on the one hand then, especially favorable if the coating of the microtiter plate with the antigen take place in a basic medium, because then, as has already been explained above, the antigens bind especially well and effectively on the microtiter plate. On the other hand, by means of the incubation with the blocking solution according to the invention, the surface of the fixed phase at the sites to which the antigen is not bound is saturated especially well and uniformly with the protein contained in the blocking solution. In this way, it very effectively prevents antigens or antibodies contained in the sample from binding to the fixed phase instead of to the antigens coupled thereto. Therefore, the non-specific background of the assay is clearly reduced.

It is recognized that it is therefore especially preferable that a microtiter plate according to the invention be provided for use in a method according to the invention.

In addition, the invention provides a test kit for verifying Aβ auto-antibodies in serum samples, including at least one antigen-coated microtiter plate, wherein the antigen is a peptide whose sequence corresponds to a sequence selected from SEQ ID No. 1, 2, or 3. It is especially favorable moreover if the test kit contains reagents already prepared for conducting the test. The experimenter, then, need only set up the appropriate serum sample to be examined as well as a appropriately adjusted control sample. It is also conceivable that the test kit already contains a selection of control samples.

It is known that the test kit preferably contains a standard, an assay buffer, a rinse solution, an enzyme conjugate, a substrate solution, and/or a stop solution.

Furthermore it is recognized that the test kit according to the invention is suitable for use in a method according to the invention.

Further features, details, and advantages of the invention result from the wording of the claims, as well as from the following description of the illustrations and embodiment examples.

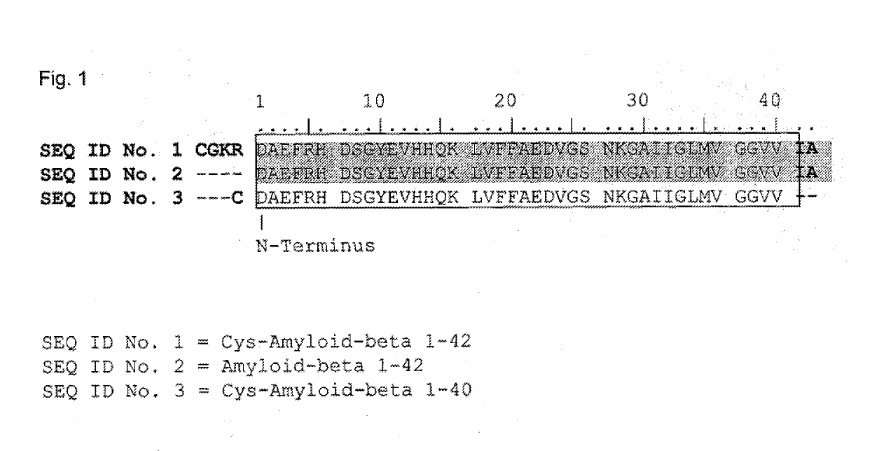

FIG. 1 Comparison of antigen sequences of embodiment examples 1 to 5,

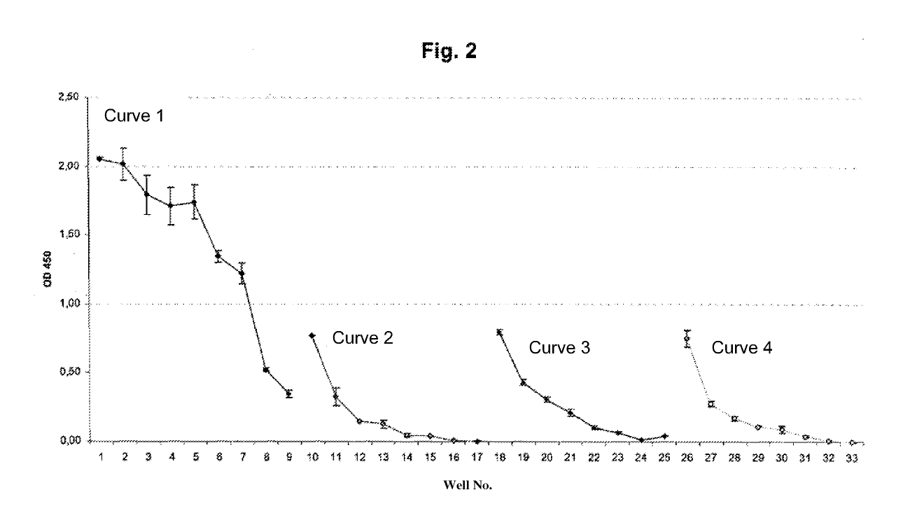

FIG. 2 OD 450 measurement values of an ELISA conducted according to embodiment example 3,

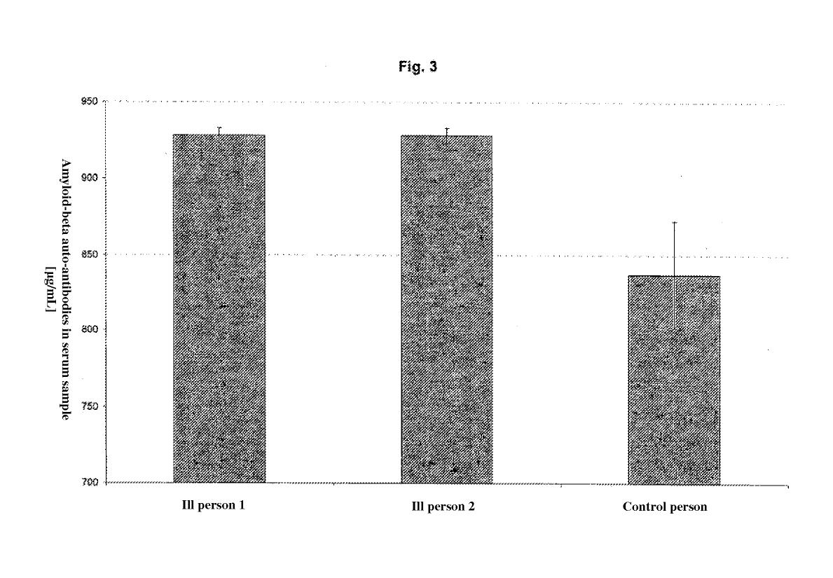

FIG. 3 amyloid-beta auto-antibody concentration in two Alzheimer patients and one healthy person, determined with the aid of the ELISA according to the invention corresponding to embodiment example 3.

It is seen in FIG. 1 that all three of the peptides can be used according to the invention for coating the fixed phase exhibit at least the amino acids 1 through 40 of the naturally occurring Aβ peptides. What is more, the boxed sequence area respectively of the sequence of the Aβ₁₋₄₀ peptide correspond, whose sequence area lies below the gray of the Aβ₁₋₄₂ peptide sequence.

The sequence denoted as Cys-amyloid-beta 1-42 and corresponding to SEQ ID No. 1 includes, besides the C-terminal, the amino acids 41 and 42 of the amyloid-beta peptide (Aβ) and, N-terminally, four additional amino acids, namely the tetrapeptide CGKR. Therefore, it involves, with the antigen corresponding to SEQ ID No. 1, a polypeptide consisting of 46 amino acids in all, which is made up of the tetrapeptide CGKR followed by the amino acids 1 through 42 of the Aβ₁₋₄₂ peptide. Printed differently is the sequence denoted by SEQ ID No. 1, a modified human peptide whose amino acids 5 through 46 represent the sequence of the naturally occurring human amyloid-beta 1-42 peptide, on which N-terminal is added the tetrapeptide Cys Gly Lys Arg, which corresponds to the amino acids 1 through 4 of SEQ ID No. 1.

It is further seen in FIG. 1 that the SEQ ID No. 2, which is denoted as amyloid-beta 1-42, corresponds to the natural sequence of the amyloid-beta 1-42 peptide (Aβ₁₋₄₂). Therefore, it involves, with the antigen corresponding to the SEQ ID No. 2, a polypeptide consisting of 42 amino acids in all, which corresponds to the amino acids 1 through 42 of the Aβ₁₋₄₂ peptide.

The SEQ ID No. 3 denoted as Cys amyloid-beta 1-40 corresponds to the natural amyloid-beta 1-40-peptide (Aβ₁₋₄₀). However the peptide is modified at the N-terminus by adding a cysteine. Therefore, it involves a polypeptide consisting of 41 amino acids in all, with the antigen corresponding to the SEQ ID No. 3, which is made up of the amino acids 1 through 40 of the Aβ₁₋₄₀ peptide and an N-terminally added cysteine. Printed differently is a modified human peptide with the sequence denoted as SEQ ID No. 3, whose amino acids 2 through 41 represent the sequence of the naturally occurring human amyloid-beta 1-40 peptide, on which the amino acid cysteine is added N-terminally, which corresponds to the amino acid 1 of the SEQ ID No. 3.

In FIG. 2, the OD 450 measurement value of a method according to the invention is seen, performed in accordance with the protocol of embodiment example 3. FIG. 3 clarifies that, using the method according to the invention, the data obtained can be invoked for a diagnosis of either Alzheimer's disease or another neurodegenerative disease.

### EMBODIMENT EXAMPLE 1

In a first embodiment example, microtiter plates are coated with an antigen corresponding to SEQ ID No. 1, that is, an Aβ peptide, which includes the amino acids 1 through 42 and additionally bears the amino acid sequence CGKR N-terminally. For this, the antigen is dissolved in a carbonate buffer with a pH of 9.6. After incubation with the coating solution thus made, the microtiter plate is incubated with a blocking solution.

Next, 250 μl of assay buffer are introduced into each well. Into each well so prepared are then placed 10 μl of sample, which involves either a standard, a control, or the actual sample to be measured, provided to the assay buffer. The sample is diluted in this way in assay buffer such that there is ultimately a volume of 260 μl diluted sample in each well.

The plate with the sample so diluted is incubated for 60 minutes in a shaker at 300 to 500 rpm at room temperature. Further, the contents of the wells are immediately shaken out and each well is washed three times with 400 μl of rinse solution. The wells are tapped off with absorbent paper to remove fluid drops remaining.

Into the rinsed wells, 50 μl of enzyme conjugate are respectively provided and incubated for 30 minutes at room temperature in a shaker at 300 to 500 rpm. Then the wells are again washed as described above and tapped off.

Then 50 μl of substrate solution are provided to each well and they are incubated for 20 minutes at room temperature. The enzyme reaction taking place at this time is ended after a 20-minute run by adding 100 μl of stop solution. Within 10 minutes of stopping the reaction, the optical density (OD) at 450±10 nm is determined for each well with a reader.

### EMBODIMENT EXAMPLE 2

The embodiment example 2 essentially corresponds to the embodiment example already described above. The wells are, however, loaded with a total volume of 200 μl of diluted sample. After removing the sample, the wells are washed respectively five times with a rinse solution, and then 100 μl of enzyme conjugate is provided into each well.

The reaction of the enzyme with the chromophore occurs during a 15-minute incubation at room temperature.

### EMBODIMENT EXAMPLE 3

In a further embodiment example, an antigen corresponding to SEQ ID No. 2, and that is, the peptide Aβ₁₋₄₂, is diluted to a concentration of 5 μg/ml in 0.1 M sodium carbonate buffer with a pH of 9.6. With this solution, a fixed phase, specifically a 96-well, high-binding ELISA plate, is incubated for two hours at 37° C. in a CO₂ incubator at 5% CO₂. Further thereto, the plate is incubated overnight with a commercially available blocking solution.

The plate so prepared is washed four times with a rinse solution according to the invention. Then appropriate standards and diluted samples are added and are incubated for 4 hours at 37° C. in a CO₂ incubator at 5% CO₂. Further, the plate is washed again four times. A PBS buffer is used as the rinse solution, which contains 0.05% of TWEEN.

For the verification of the Aβ auto-antibodies bound on the plate, an HRP-coupled, anti-human, second antibody is used. This is diluted to a ratio of 1:4000 in the previously used blocking solution. TMNB is used as the chromogenic substrate. The color reaction is stopped using sulfuric acid and is evaluated at a wavelength of 450 nm by determining the OD.

In the evaluation, the OD of the standard is determined first and then compared to the sample or control. A comparison curve is determined for this with the aid of an established series of standard concentrations. The samples and controls are each applied several times and in different dilutions.

The loading of a microtiter plate can be seen as follows, by way of example:
Wells 1 to 8: Standard in different concentrations for setting up a comparison curve
Wells 9 to 16: Control-sample from a person without disease, in different dilutions
Wells 17 to 24: Samples to be measured from a possibly ill person in different dilutions

The respective design of the test can moreover be varied as needed. So for a verification method carried out according to embodiment example 3, by way of example, 40 microtiter-plate wells in all to be measured are loaded as represented in Table 1. What is more, two ill persons and one control person whose age corresponds to the age of ill person No. 1 were studied. One serum sample each was taken from all three persons for this.

The first 16 wells were loaded with a standard dilution series in order to set up a comparison curve, using which the concentration of Aβ auto-antibody present in the samples can then be calculated using the OD measurement values of the samples.

A mixture of Aβ auto-antibodies naturally occurring in the serum was used as a standard, which was made from a commercially available product for the intravenous administration of immunglobulin.

TABLE 1
Standard:
Aβ auto-antibody from IVIg in PBS

| Well No. | Concentration | | Well No. | Concentration | |
|---|---|---|---|---|---|
| 1 | 2000 | μg/ml | 9 | 25 | μg/ml |
| 2 | 1000 | μg/ml | 10 | 12.5 | μg/ml |
| 3 | 500 | μg/ml | 11 | 5 | μg/ml |
| 4 | 250 | μg/ml | 12 | 2.5 | μg/ml |
| 5 | 125 | μg/ml | 13 | 1.25 | μg/ml |
| 6 | 100 | μg/ml | 14 | 1 | μg/ml |
| 7 | 75 | μg/ml | 15 | 0.5 | μg/ml |
| 8 | 50 | μg/ml | 16 | | |

| III Person No. | | III Person No. 2 | | Control person | |
|---|---|---|---|---|---|
| Serum sample | | Serum sample | | Serum sample | |
| Well No. | Dilution | Well No. | Dilution | Well No. | Dilution |
| 17 | 1:1000 | 25 | 1:1000 | 33 | 1:1000 |
| 18 | 1:5000 | 26 | 1:5000 | 34 | 1:5000 |
| 19 | 1:10,000 | 27 | 1:10,000 | 35 | 1:10,000 |
| 20 | 1:25,000 | 28 | 1:25,000 | 36 | 1:25,000 |
| 21 | 1:50,000 | 29 | 1:50,000 | 37 | 1:50,000 |
| 22 | 1:100,000 | 30 | 1:100,000 | 38 | 1:100,000 |
| 23 | 1:500,000 | 31 | 1:500,000 | 39 | 1:500,000 |
| 24 | 1:1,000,000 | 32 | 1:1,000,000 | 40 | 1:1,000,000 |

Loading plan for 40 wells of a microtiter plate according to the invention:
Wells 1 to 16: Standard concentration series for setting up a comparison curve
Wells 17 to 24: Dilution series for a serum sample of a first ill person
Wells 25 to 32: Dilution series for a serum sample of a second ill person
Wells 33 to 40: Dilution series for a serum sample of a healthy control person

The wells of the microtiter plate loaded as represented in Table 1 with sample or standard were incubated according to the protocol described above for 4 hours at 37° C., then washed, incubated with enzyme conjugate, washed again, and incubated with substrate solution. After stopping the enzyme-substrate reaction, the OD 450 was determined with an appropriate reader.

The measurement values of such an OD 450 determination are represented in FIG. 2a. Curve 1 shows the OD measurement values of the wells loaded with the standard series, curve 2 the measurement values of the diluted serum samples of ill person 1, curve 3 the measurement values of the diluted serum samples of ill person 2, and curve 4 the measurement values of the diluted serum samples of the control person.

By comparing the measurement values of the ill persons or of the control person with the measured standard values of a known antibody concentration, the concentrations of Aβ auto-antibodies in the respective serum samples can be further calculated in measuring from the OD 450 values.

In FIG. 2b, it is seen that both ill persons each exhibit more than 900 μg/ml of Aβ auto-antibodies in serum. The healthy control person displays, in contrast, a clearly lower value of only 840 μg/ml.

### EMBODIMENT EXAMPLE 4

In a further embodiment example, 96-well ELISA plates were coated with an antigen corresponding to SEQ ID No. 3, that is, Aβ₁₋₄₀ peptide, which is provided N-terminally with a cysteine.

Here the ELISA plates were incubated at 4° C. overnight with a coating solution. To make the coating solution, 5 μg/ml of antigen were dissolved immediately before incubation in a 100 mM sodium bicarbonate buffer with a pH of 9.6.

Further, the plates were incubated with blocking solution for 2 hours at room temperature, or alternatively, at 4° C. overnight. A commercially available solution was used as a blocking solution, for example SuperBlock from the Pierce Company, Germany.

To set up standards, human IVIgG, affinity-purified auto-antibodies were diluted with salt solution that has a pH of 7.4, such that they were in concentrations of 0.78 to 50 μg/ml. The standards so made were incubated at 4° C. overnight on the antigen-coated plates.

An HRP-conjugated, α-human, antibody from goats was used as a second antibody, which specifically recognized the heavy and light chains of immunglobulins (HRP-labeled, H&L, chain-specific goat, anti-human, from Calbiochem, Darmstadt, Germany). This was used in a dilution of 1:3000. The incubation took place for one hour at 37° C. 3,3′,5,5′-tetramethylbenzidine (TMB) was used as a chromogenic substrate. The enzyme reaction was terminated with 2N sulfuric acid (H₂SO₄). The plates were evaluated at 50 nm using a reader from Thermo Electron Corp.

The washing step, as well as the enzyme reaction, take place in this embodiment example in accordance with the embodiment examples described above.

### EMBODIMENT EXAMPLE 5

In a further embodiment variant, the invention includes a test kit for performing an ELISA according to the invention. The test kit contains a microtiter plate which is coated with a peptide that has a sequence corresponding to SEQ ID No. 1, and the immobilized binding partner for the Aβ auto-antibody to be verified is the antigen. In the test kit are, in addition, a blocking solution, a rinse solution, an assay buffer, a concentrated amount of standard, an enzyme conjugate, a substrate, and a stop solution.

The invention is not limited to one of the previously described embodiments, but can be modified in numerous ways.

It is recognized that in one method, in particular an enzyme-linked immunosorbent assay (ELISA), for the in-vitro verification of Aβ auto-antibodies in human blood serum and/or plasma, comprising the steps of the preparation of an antigen-coated fixed phase, incubation of the fixed phase with a blocking solution, incubation of the fixed phase with a sample to be examined, immunological verification of the Aβ auto-antibody in the fixed phase, and readout of the verification results in the fixed phase using a reader, it is advantageous if the preparation of the antigen-coated fixed phase includes the incubation of the fixed phase with a coating solution in which an antigen is dissolved which exhibits a peptide sequence selected from the group of SEQ ID No. 1, SEQ ID No. 2, or SEQ ID No. 3. At the same time, it is especially favorable if the antigen preferably exhibits a peptide sequence corresponding to SEQ ID No. 1. In addition, it is preferable if the coating solution is a carbonate buffer with a basic pH value and if the blocking solution is a Tris-buffered protein solution with a pH of 7.4 containing at least one antimicrobial agent. The incubation of the fixed phase with the coating solution takes place at 4° C. overnight or at 37° C. and 5% CO₂ for 2 hours. The incubation with the blocking solution is performed at 4° C. overnight.

It is preferred that the fixed phase be a microtiter plate and the incubation of the microtiter plate with the solution to be examined comprise the following steps: introduction of a diluted sample into each well of the microtiter plate, whereby the introduction of the diluted sample occurs such that a total volume of 200 to 300 μl of assay buffer containing the sample is then present in each well, incubation for 60 minutes in a shaker at 300 to 500 rpm and room temperature.

The immunological verification of the Aβ auto-antibody comprises the following steps: tapping the contents out of the wells, three to five washings of the wells with 300 to 500 μl respectively of rinse solution per well, removal of remaining fluid drops by wiping the wells with an absorbent paper, introducing 50 μl to 100 μl of enzyme conjugate into each well, incubating for 30 to 60 minutes in a shaker at 300 to 500 rpm and room temperature, shaking the contents out of the wells, washing the wells three to five times with 300 to 500 μl respectively of rinse solution per well, removing remaining fluid drops by wiping the wells with an absorbent paper, adding 50 μl substrate solution into each well, incubating 15 to 20 minutes at room temperature, stopping the enzyme reaction by adding 100 μl of stop solution into each well.

The readout of the verification results takes place at 450±10 nm, preferably within 10 minutes of adding the stop solution.

It is favorable if the assay buffer is a sodium phosphate buffer with a pH of 7.0, including 3 to 9% BSA, 0.01 to 3% TWEEN, and at least one preservative selected from the group of 5-bromo-5-nitro-1,3-dioxane (BND), 2-chloroacetamide (CAA), 2-hydroxypyridine-N-oxide (HPO), N-methylisothiazolone (MIT), sodium azide, Thimerosal, and ProClin, if the rinse solution is a Tris-buffer containing 0.01 to 3% TWEEN, if the enzyme conjugate contains an enzyme-conjugated antibody directed against human IgG which is selected from a species originating from goat, mouse, porpoise, rat, donkey, cow, and sheep, in which the enzyme conjugated with the antibody is selected from the group of horseradish peroxidase (HRP), alkali phosphatase (AP), beta-galactosidase (β-gal), if the substrate solution contains at least one chromophore selected from the group of 3,3′-diaminobenzidine (DAB), 3-amino-9-ethylcarbazole (AEC), 4-chloro-1-naph-thol (CN); 3,3′,5,5′-tetramethylbenzidine (TMB), new fuchsine, naph-thol-AS-MX-phosphate, 5-bromo-5-chloro-3-indoxyl phosphate (BLIP), nitro blue tetrazolium chloride (NBT), 5-bromo-4-chloro-3-indoxyl-β-D-galactopyranoside (X-gal), 5-bromo-3-indolyl-β-D-galactopyranoside (blue-gal), 6-chloro-3-indolyl-β-D-galactopyranoside (Y-gal), 5-iodo-3-indolyl-β-D-galactopyranoside (purple-gal), 5-bromo-6-chloro-3-indolyl-β-D-galactopyranoside (magenta-gal), N-methyl-indolyl-β-D-galactopyranoside (green-gal), 4-methylumbelliferyl-β-D-galacto-pyranoside (MUG), in soluble form, which displays a color reaction upon reaction with the enzyme of the enzyme conjugate, and if the stop solution has a pH value lower than 7.0 and preferably is a dilute hydrochloric or sulfuric acid.

It is recognized that it is preferable, with a microtiter plate for verification of Aβ auto-antibodies with at least one well, that each well be coated with a peptide corresponding to one of the sequences SEQ ID No. 1 to 3. Preferably, the microtiter plate exhibits at least one test unit comprising 24 wells. At the same time, the microtiter plate most especially preferably exhibits a plurality of test units which can be used independently of one another. What is more, the wells can have a round, flat, V-shaped, or C-shaped bottoms. A microtiter plate according to the invention is made according to a method including the following steps: preparation of an uncoated microtiter plate, incubation of the microtiter plate with a coating solution which consists of a basic carbonate buffer with a pH of 9.6 in which are dissolved 5 μg/ml of an antigen corresponding to one of the sequences according to SEQ ID No. 1, 2, or 3 for two hours at 37°, and incubation of the microtiter plate with a blocking solution containing a Tris-buffered protein solution with a pH of 7.4 containing at least one antimicrobial agent at 4° C. overnight.

The use of a microtiter plate according to the invention is appropriate in a method according to the invention.

It is recognized, in addition, that a test kit for verification of Aβ auto-antibodies in serum samples, including at least one antigen-coated microtiter plate in which the antigen is a peptide whose sequence corresponds to a sequence selected from SEQ ID No. 1, 2, or 3, is an advantageous embodiment of the invention. At the same time, it is favorable if the test kit contains an assay buffer, a rinse solution, an enzyme conjugate, a substrate solution, and/or a stop solution. The test kit according to the invention can be used advantageously in a method according to the invention.

All the features and advantages which emerge from the claims, the description, and the drawings, including constructive details, spatial dispositions, and steps of the method, can be essential to the invention both by themselves and in various combinations.

### REFERENCE LIST

| SEQ ID No. 1 | Cys-amyloid-beta 1-42 |
|---|---|
| SEQ ID No. 2 | Amyloid-beta 1-42 |
| SEQ ID No. 3 | Cys-amyloid-beta 1-40 |
| Curve 1 | Standard |
| Curve 2 | III person 1 |
| Curve 3 | III person 2 |
| Curve 4 | control person |
| N-terminus | N-terminal amino acid of the natural Aβ-peptide |
| 1 | Amino acid No. 1 of the natural Aβ |
| 10 | Amino acid No. 10 of the natural Aβ |
| 20 | Amino acid No. 20 of the natural Aβ |
| 30 | Amino acid No. 30 of the natural Aβ |
| 40 | Amino acid No. 40 of the natural Aβ |

### ABBREVIATIONS

| | |
|---|---|
| Aβ | Amyloid-beta |
| Aβ₁₋₄₀ | Amyloid-beta peptide comprising the amino |
| | acids 1 to 40 |
| Aβ₁₋₄₂ | Amyloid-beta peptide comprising the amino |
| | acids 1 to 42 |
| AEC | 3-amino-9-ethylcarbazole |
| AP | Alkali phosphatase |
| APP | Amyloid precursor protein |
| BCIP | 5-bromo-5-chloro-3-indoxyl phosphate |
| Blue-gal | Bromo-3-indolyl-β-D-galactopyranoside |
| BND | 5-bromo-5-nitro-1,3-dioxane |
| CAA | 2-chloroacetamide |
| CN | 3-chloro-1-naphthol |
| CSF | Cerebrospinal fluid (liquor) |
| β-gal | Beta-galactosidase |
| Green-gal | N-methylindolyl-β-D-galactopyranoside |
| DAB | 3,3′-diaminobenzidine |
| ELISA | Enzyme-linked immunosorbent assay |
| HPO | 2-hydroxypyridine-N-oxide |
| HRP | Horseradish peroxidase |
| α-human IVIgG | Intravenously administrable immunoglobulin |
| Magenta-gal | 5-bromo-6-chloro-3-indolyl-β-D-galactopyranoside |
| MIT | N-methylisothiazolone |
| MUG | 4-methyl-umbelliferyl-β-D-galactopyranoside |
| NBT | Nitro blue tetrazolium chloride |
| OD | Optical density |
| OD 450 | Optical density at 450 nm |
| Purple-gal | 5-iodo-3-indolyl-β-D-galactopyranoside |
| rpm | Revolutions per minute |
| TMB | 3,3′,5,5′-tetramethylbenzidine |
| X-gal | 5-bromo-4-chloro-3-indoxyl-β-D-galactopyranoside |
| Y-gal | 6-chloro-3-indolyl-β-D-galactopyranoside |

### SEQUENCE PROTOCOL-FREE TEXT

Modified Human Peptide

N-terminally added tetrapeptide sequence Cys Gly Lys Arg

Sequence of the naturally occurring human amyloid-beta 1-42 peptide Cysteine

Sequence of the naturally occurring human amyloid-beta 1-40 peptide

1. A method for the in-vitro verification of amyloid-beta auto-antibodies in human blood serum and/or plasma, as well as CSF, comprising the steps
a) preparation of an antigen-coated fixed phase,
b) incubation of the fixed phase with a blocking solution,
c) incubation of the fixed phase with a sample to be examined,
d) immunological verification of the amyloid-beta auto-antibody in the fixed phase, and
e) readout of the verification results from the fixed phase using a reader,
characterized in that, the preparation of the antigen-coated fixed phase includes the incubation of the fixed phase with a coating solution in which an antigen is dissolved, which exhibits a peptide sequence selected from the group consisting of SEQ ID No. 1, SEQ ID No. 2, and SEQ ID No. 3.

2. The method according to claim 1, characterized in that the antigen exhibits a peptide sequence corresponding to SEQ ID No. 1.

3. The method according to claim 1, characterized in that the coating solution is a carbonate buffer with a basic pH value.

4. The method according to claim 1 characterized in that the blocking solution is a Tris-buffered protein solution with a pH of 7.4 containing at least one antimicrobial agent.

5. The method according to claim 1 characterized in that the incubation of the fixed phase with the coating solution takes place at 4° C. overnight.

6. The method according to claim 1 characterized in that the incubation of the fixed phase with the coating solution takes place at 37° C. and 5% CO₂ for 2 hours.

7. The method according to claim 1 characterized in that the incubation with the blocking solution is performed at 4° C. overnight.

8. The method according to claim 1 characterized by the fact that the fixed phase is a microtiter plate and that the incubation of the microtiter plate with the solution to be examined comprises the following steps
a) introduction of a diluted sample into each well of the microtiter plate, in which the introduction of the diluted sample takes place such that a total volume of 200 to 300 μl of assay buffer containing the sample is then present in each well,
b) incubation for 60 minutes in a shaker at 300 through 500 rpm and room temperature.

9. The method according to claim 1 characterized in that the assay buffer is a sodium phosphate buffer with a pH of 7.0 including
a) 3 to 9% BSA,
b) 0.01 to 3% of a polysorbate
c) as well as at least one preservative selected from the group consisting of 5-bromo-5-nitro-1,3-dioxane (BND), 2-chloroacetamide (CAA), 2-hydroxypyridine-N-oxide (HPO), N-methylisothiazolone (MIT), sodium azide, Thimerosal, and a biozide containing isothiazolones.

10. The method according to claim 1 characterized in that
the immunological verification of the amyloid-beta auto-antibody includes
the following steps
a) tapping the content out of the wells
b) three to five washings of the wells with 300 to 500 μl respectively of rinse solution per well
c) removing remaining fluid drops by wiping the wells with an absorbent paper
d) introducing 50 μl to 100 μl enzyme conjugate into each well
e) 30-to-60-minute incubation in a shaker at 300 to 500 rpm and room temperature
f) shaking the contents out of the wells
g) three to five washings of the wells with 300 to 500 μl respectively of rinse solution per well
h) removing remaining fluid drops by wiping the wells with an absorbent paper
i) adding 50 μl of substrate solution into each well
j) incubating 15 to 20 minutes at room temperature
k) stopping the enzyme reaction by adding 100 μl of stop solution into each well.

11. The method according to claim 1 characterized in that the rinse solution is a Tris-buffer containing 0.01 to 3% of a polysorbate.

12. The method according to claim 1 characterized in that the readout of the verification results takes place at 450±10 nm

13. The method according to claim 1 characterized in that the readout takes place within 10 minutes of adding the stop solution.

14. The method according to claim 1 characterized in that the enzyme conjugate contains an enzyme-conjugated antibody directed against human IgG, which originates from a species selected from goat, mouse, porpoise, rat, donkey, cow, and sheep, in which the enzyme conjugated with the antibody is selected from the group of horse-radish peroxidase (HRP), alkali phosphatase (AP), beta-galactosidase (β-gal).

15. The method according to claim 1 characterized in that the substrate solution contains at least one chromophore selected from the group consisting of 3,3′-diaminobenzidine (DAB), 3-amino-9-ethylcarbazole (AEC), 4-chloro-1-naphthol (CN); 3,3′,5,5′-tetramethylbenzidine (TMB), new fuchsine, naphthol-AS-MX-phosphate, 5-bromo-5-chloro-3-indoxyl phosphate (BCIP), nitro blue tetrazolium chloride (NBT), 5-bromo-4-chloro-3-indoxyl-β-D-galactopyranoside (X-gal), 5-bromo-3-indolyl-β-D-galactopyranoside (blue-gal), 6-chloro-3-indolyl-β-D-galacto-pyranoside (Y-gal), galactopyranoside (purple-gal), 5-bromo-6-chloro-3-indolyl-β-D-galactopyranoside (magenta-gal), N-methylindolyl-β-D-galactopyranoside (green-gal), 4-methylumbelli-feryl-β-D-galactopyranoside (MUG), in soluble form, which displays a color reaction upon reaction with the enzyme of the enzyme conjugate.

16. The method according to one claim 1 characterized in that the stop solution has a pH value lower than 7.0.

17. A microtiter plate with at least one well, characterized in that each well is coated with a peptide corresponding to one of the sequences SEQ ID No. 1 to 3.

18. The microtiter plate according to claim 17, characterized in that the microtiter plate exhibits at least one test unit comprising 24 wells.

19. The microtiter plate according to claim 18, characterized in that the microtiter plate exhibits a plurality of test units, which can be used independently of one another.

20. The microtiter plate according to claim 17 characterized in that the wells have a round, flat, V-shaped, or C-shaped bottom.

21. The microtiter plate according to claim 1 characterized in that it is made according to a method comprising the following steps
a) preparation of an uncoated microtiter plate
b) incubation of the microtiter plate with a coating solution which consists of a basic carbonate buffer with a pH of 9.6, in which is dissolved 5 μg/ml of an antigen corresponding to one of the sequences according to SEQ ID No. 1, 2, or 3 for two hours at 37°, and
c) incubation of the microtiter plate with a blocking solution containing a Tris-buffered protein solution with a pH of 7.4 containing at least one antimicrobial agent, at 4° C. overnight.

22. The microtiter plate according to claim 1 for use in a method corresponding to claim 1.

23. A test kit for verifying amyloid-beta auto-antibodies in blood serum/plasma and/or CSF samples, including at least one antigen-coated microtiter plate, characterized in that the antigen is a peptide whose sequence corresponds to a sequence selected from SEQ ID No. 1, 2, or 3.

24. The test kit according to claim 23, characterized in that the test kit contains an assay buffer, a rinse solution, an enzyme conjugate, a substrate solution, and/or a stop solution.

25. The test kit according to claim 23 for use in a method corresponding to claim 1.

26. The method of claim 1 wherein the method utilizes an enzyme-linked immunosorbent assay (ELISA).

27. The method of claim 16 wherein the stop solution is a dilute hydrochloric or sulfuric acid solution.
